(19) United States
(12) Patent Application Publication
(10) Pub. No.: US 2022/0323047 A1
(43) Pub. Date: Oct. 13, 2022

(54) BIOLOGICAL FLUID TEST DEVICE, IN PARTICULAR A SALIVA TEST DEVICE

(71) Applicant: TODA GROUPE, STRASBOURG (FR)

(72) Inventor: Yossi BERROS, STRASBOURG (FR)

(21) Appl. No.: 17/639,810

(22) PCT Filed: Sep. 3, 2020
(86) PCT No.: PCT/FR2020/000234

§ 371 Date: Mar. 2, 2022

(30) Foreign Application Priority Data
Sep. 3, 2019 (FR) FR1909663

(51) Int. Cl. A61B 10/00

(52) U.S. Cl. CPC A61B 10/0051; A61B 10/0096; A61B 2010/0006; A61B 2010/0009

(57) ABSTRACT

The invention relates to a biological fluid test device (1), in particular a saliva test device, the device comprising:—a sample collector (2) in the form of a membrane, —a means for checking whether the quantity is sufficient, the collector being configured to cause a biological fluid to migrate to the checking means, - a means for displaying, on the device, a test result for at least one desired substance, characterised in that the device (1) comprises, or is associated with, at least one means for removing the collector and storing it for second expert opinion purposes. The invention also relates to a corresponding kit and method.

The present invention relates to the field of biological fluid tests, in particular saliva tests, for detecting the presence of particular substances such as drugs, viruses, bacteria, etc.

Currently on the market, it is necessary, after performing a drugs test which proves to be positive, to confirm the results obtained by means of a second opinion solution.

In this case, it will be necessary to again sample the saliva in the mouth of the person tested (subject) using a second sampling system in order to submit it for verifying the results.

However, the fact of taking a second sample is uncomfortable for the patient and for the person performing it, and in addition, when taking the second sample the sample quantity may exhibit some deterioration due to a number of factors, and in particular due to the fact that the patient is not inclined for this sample to be usable.

An object of the present invention is that of proposing biological fluid testing means which limits the complications associated with a second sample for a second opinion, in particular the fact that taking the new sample is uncomfortable, the risk of deterioration, and the risk of the subject failing to collaborate.

In order to achieve this object, the invention proposes a biological fluid test device, in particular a saliva test device, the device comprising:
a sample collector in the form of a membrane,
a means for checking whether the quantity is sufficient, the collector being configured to cause a biological fluid to migrate to the checking means,
a means for displaying, on the device, a test result for at least one desired substance.

According to a first aspect, the device comprises, or is associated with, at least one means for removing the collector and storing it for the purpose of a second opinion.

Advantageously, the invention involves a simplified solution for collecting and preserving the saliva sample in order to take a second opinion.

According to other aspects taken in isolation or combined according to any technically possible combinations:
the means for checking whether the quantity is sufficient comprises a control line; and/or
the means for checking whether the quantity is sufficient comprises an indicator; and/or
the means for displaying a test result comprises a window on the device, associated with at least one revealing means of the device; and/or
the means for removing the collector comprises pincers; and/or
the means for removing the collector comprises an automatic ejection system; and/or
the means for removing the collector comprises a scored part of the collector; and/or
the means for storing the collector comprises a specific preservation solution and/or a specific container;
the means for storing the collector comprises a container comprising a preservation solution.

The invention furthermore relates to a biological fluid test kit, in particular a saliva test kit, the kit comprising:
the test device according to the invention, and
at least one means for removing the collector and/or for storing it for the purpose of a second opinion, in particular a specific container, and/or a specific preservation solution, or means for producing the specific preservation solution.

Another object of the invention relates to a method for performing a biological fluid test, in particular a saliva test, and a second opinion if the test is positive, the method comprising the steps of:
performing a saliva test by means of a test device according to the invention or a kit according to the invention,
if the test is positive, removing the collector in the form of a membrane, and
storing the collector in the form of a membrane for the purpose of a second opinion.

More generally, the invention also relates to a method for performing a saliva test, and a second opinion if the saliva test is positive, the method comprising the steps of:
performing a saliva test by means of a saliva test device comprising a sample collector in the form of a membrane, which comprises a means for checking whether the quantity is sufficient, and a means for displaying, on the device, a saliva test result,
if the test is positive, removing the collector in the form of a membrane, and
storing the collector in the form of a membrane for the purpose of a second opinion.

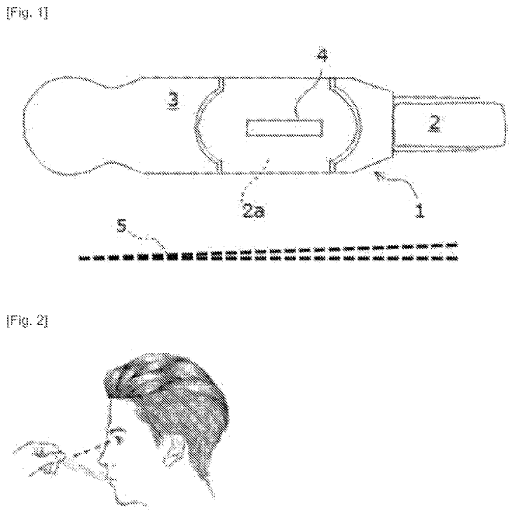

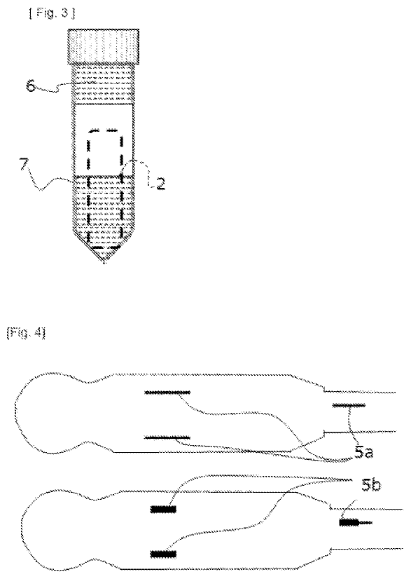

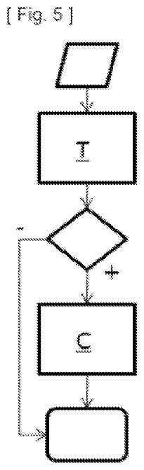

The invention will be described in greater detail by way of the description of non-limiting embodiments, and with reference to the accompanying drawings which illustrate variants of the invention, in which:

[Fig. 1] is a plan view of a saliva test device according to a preferred embodiment of the invention, associated with a first embodiment of means for removing the collector;

[Fig. 2] illustrates a saliva sample being taken by means of the device of FIG. 1;

[Fig. 3] is a plan view of a preservation container comprising a collector of a device according to FIG. 1;

[Fig. 4] illustrates a second embodiment of an ejection means of the collector of a device according to an embodiment; and

[Fig. 5] shows an outline of the method for performing the test and the preservation for a second opinion.

The invention relates to the biological fluid tests for detecting the presence of particular substances such as drugs, viruses, bacteria, etc.

These tests are preferably designed to be performed on human subjects. In the present case, these are saliva tests, but tests on other biological fluids, such as urine, may also be envisaged on the basis of the same principle.

The invention is applicable in the saliva test procedures where a second opinion is required, without needing to re-take a second sample, thus preventing the associated complications (uncomfortable new sampling, risk of deterioration/failure to collaborate, etc.).

This type of application can be illustrated by part of FIG. 5. In essence, a saliva test is performed on a subject in a first step T. If the test is positive, a second opinion (C) must be taken, for example by means of more advanced analyses.

Thus, the invention relates to a biological fluid test device 1. This is in particular a saliva test device.

The device comprises a sample collector 2 in the form of a membrane. The collector 2 may be substantially planar in shape. This makes it possible to limit the bulk of the device in the region of the collector, and facilitates the storage thereof.

The collector 2 comprises an absorbent material which makes it possible to retain the sampled biological fluid and to cause it to migrate, for example by capillary action.

The collector is associated with a body 3 of the device comprising a portion for migration of the biological fluid 2a, in particular of the saliva. This is in particular a housing containing at least one migration strip for the biological fluid. The collector 2 is for example in contact with the migration strip 2aof the body 3 of the device 1. It is possible to envisage a collector 2 which is integral with the strip 2a.

The device 1 further comprises a means for checking whether the quantity is sufficient. This is in particular one or more elements of the device, more particularly of the body 3 of the device, which make it possible to signal, to the user, that a sufficient quantity of fluid has been sampled.

The collector 2 is thus configured to cause the biological fluid to migrate to the checking means.

According to a variant, the means for checking whether the quantity is sufficient comprises a control line. This is for example an adsorbed system which reacts upon contact of the biological fluid to reveal a colored line, for example depending on the pH, of a specific reagent of the biological fluid, such as amylase, or of a system of recombinant immunoglobulins.

Alternatively or in combination, the means for checking whether the quantity is sufficient comprises an indicator. This is for example a colored indicator that is sensitive to the pH of the biological fluid or to a specific reagent of the biological fluid.

The device 1 further comprises a means for displaying a test result 4 for at least one desired substance. This is in particular a window 4 on the device 1, in particular on the housing and facing the migration strip 2a.

The window 4 is associated with at least one revealing means of the device 1, in particular on the migration strip.

This is for example an adsorbed system which reacts upon contact of the desired substance to reveal a colored line, for example on the basis of a reaction with a specific reactive function of the desired substance, or of a system of recombinant immunoglobulins.

The display makes it possible to have a first test result without having to remove the collector 2 to perform advanced analyses, and to do this if the first result is positive.

According to the invention, the device 1 is associated with at least one means for removing the collector 2 in order to store it for the purpose of a second opinion.

In a variant, this is for example pincers 5 which make it possible to grasp the collector 2 and to remove it, for example by pulling towards the outside.

In another variant, this is a cutting means such as scissors or a scalpel, preferably associated with a scored portion of the collector.

Alternatively or in combination, according to the invention, the device comprises a means for removing the collector in order to store it for the purpose of a second opinion. This is for example a system for automatic ejection of the collector 5a, 5b. This type of system is designed to cause the collector to move out.

FIG. 4 shows an automatic ejection means. The top figure shows the rear face of the device which comprises piercings and slits 5a for fitting an ejection base. The bottom figure shows the base 5b coming to fit into the rear of the device 1, in the piercing and slits 5a, in order to eject the collector by means of sliding.

The collector 2 thus removed is then stored for example in an appropriate medium. This is for example a specific buffer solution 6 which makes it possible to prevent deterioration of the sample. The makeup of this solution depends on the biological fluid in question and the desired substance.

The preservation solution 6 may be optimized in order to favor the extraction of the desired compounds.

The collector 2 may be stored in a specific container 7, which for example does not allow light to pass through. This makes it possible to prevent deterioration of desired compounds that are light-sensitive.

Advantageously, the invention involves a simplified solution for collecting and preserving the saliva sample in order to take a second opinion. The collector 2 may be sufficiently preserved, so as to prevent deterioration of the sample, and to optimize the extractions. Good extraction of the desired compounds makes it possible to improve the subsequent advanced tests.

The invention avoids the need to press a collector, applying large forces, because this does not make it possible to extract a satisfactory quantity of desired compounds trapped in the collector 2.

The invention furthermore relates to a corresponding biological fluid test kit. This is in particular a saliva test kit.

The kit comprises a test device 1 as described above, and at least one means as described above for removing the collector 2 and storing it for the purpose of a second opinion.

In a variant, this is a specific preservation solution or means for producing the specific preservation solution 6. For example, the kit includes a composition to be solubilized using distilled water.

In a variant, a specific container 7 can be provided which prevents the passage of light, for example.

The invention furthermore relates to a corresponding method for performing a biological fluid test. This is in particular a method for performing a saliva test, and a second opinion if the test is positive.

The method comprises a step (T) for performing a saliva test by means of a test device as described above, or a kit as described above.

The rest of the method depends on the result of the test; positive (+) or negative (−).

If the test is negative, the method ends.

If the test is positive, the method further comprises a step of removing the collector in the form of a membrane 2, in particular using a means as described above.

The method further comprises a step (C) of storing the collector in the form of a membrane 2 for the purpose of a second opinion, in particular using a means as described above.

If a second opinion is taken, the container 7 may be used for performing more advanced tests.

1. Biological fluid test device , in particular a saliva test device, the device comprising:
a sample collector in the form of a membrane,
a means for checking whether the quantity is sufficient, the collector being configured to cause a biological fluid to migrate to the checking means,
a means for testing for at least one desired substance,
a means for displaying, on the device, a test result for at least one desired substance, characterized in that the device comprises, or is associated with, at least one means for removing the collector and storing it for the purpose of more advanced second opinion analysis.

2. Test device according to claim 1, characterized in that the test means a migration strip which is in contact with or integral with the collector.

3. Test device according to claim 1, characterized in that the means for checking whether the quantity is sufficient comprises an indicator and/or a control line.

4. Test device according to claim 1, characterized in that the means for displaying a test result comprises a window on the device, associated with at least one revealing means of the device.

5. Test device according to claim 1, characterized in that the means for removing the collector comprises pincers.

6. Test device according to claim 1, characterized in that the means for removing the collector comprises an automatic ejection system.

7. Test device according to claim 1, characterized in that the means for removing the collector comprises a scored part of the collector.

8. Test device according to claim 1, characterized in that the means for storing the collector comprises a specific preservation solution forming a suitable medium, depending on the biological fluid in question and a desired substance, and/or a specific container of a desired compound.

9. Biological fluid test kit, in particular a saliva test kit, the kit comprising:
a test device according to claim 1, and
at least one means for removing the collector and/or for storing it for the purpose of a second opinion, in particular a specific container, and/or a specific preservation solution, or means for producing the specific preservation solution.

10. Method for performing a biological fluid test, in particular a saliva test, and a second opinion if the test is positive, the method comprising the steps of:
performing a saliva test using a test device according to claim 1,
if the test is positive, removing the collector in the form of a membrane, and
storing the collector in the form of a membrane for the purpose of a second opinion.
